(19) United States
(12) Patent Application Publication
(10) Pub. No.: US 2020/0338039 A1
(43) Pub. Date: Oct. 29, 2020

(54) METHODS AND MATERIALS FOR ASSESSING BIOLOGICAL AGE AND SLOWING THE PROGRESS OF EXCESSIVE BIOLOGICAL AGING

(71) Applicants: The Scripps Research Institute, La Jolla, CA (US); Mayo Foundation for Medical Education and Research, Rochester, MN (US)

(72) Inventors: Laura Jane Niedernhofer, Minneapolis, MN (US); Matthew James Yousefzadeh, St. Paul, MN (US); Nathan Kyle LeBrasseur, Rochester, MN (US); Marissa Joy Schafer, Rochester, MN (US)

(21) Appl. No.: 16/768,615

(22) PCT Filed: Nov. 30, 2018
(86) PCT No.: PCT/US2018/063485

§ 371 Date: May 29, 2020

Related U.S. Application Data
(60) Provisional application No. 62/593,395, filed on Dec. 1, 2017.

(51) Int. Cl. A61K 31/352; A61P 43/00

(52) U.S. Cl. CPC A61K 31/352; A61P 43/00

(57) ABSTRACT

This document provides methods for determining biological age of mammalian subjects or assessing whether the subjects are aging normally. Also provided herein are methods for determining whether therapeutic treatment or other interventions for reversing or slowing down aging are effective. This document also provides methods and materials for slowing the progression of biological aging.

### CROSS-REFERENCE TO RELATED APPLICATIONS

This application claims the benefit of U.S. Patent Application Ser. No. 62/593,395, filed on Dec. 1, 2017. The disclosure of the prior application is considered part of (and is incorporated by reference in) the disclosure of this application.

### STATEMENT REGARDING FEDERAL FUNDING

This invention was made with government support under AG043376 awarded by the National Institutes of Health. The government has certain rights in the invention.

### BACKGROUND

### 1. Technical Field

This document relates to methods and materials for assessing biological age. For example, this document provides methods and materials for assessing the biological age of a mammal by determining the MCP-1 polypeptide levels within the mammal. This document also relates to methods and materials for slowing the biological aging of a mammal (e.g., a mammal having excessive biological aging based on MCP-1 polypeptide levels as compared to the chronological age of the mammal).

### 2. Background Information

Aging is the major risk factor for numerous chronic diseases and is responsible for the bulk of health care costs (Goldman et al. 2013 Health Aff (Millwood). 32:1698-1705).

The fastest growing segment of the world population is the elderly, causing an exponential rise in the incidence of chronic diseases. To address this healthcare crisis, there is a growing interest in identifying ways to therapeutically target aging in order to prevent, delay or attenuate multiple age-related diseases simultaneously (Burd et al. 2016 J Gerontol A Biol Sci Med Sci. 71:1388-1394). A number of therapeutic strategies have emerged (Harrison et al. 2009 Nature. 460:392-395; Zhu et al. 2015 Aging cell. 14:644-658; Barzilai et al. 2016 Cell Metab. 23:1060-1065). However, a major barrier to clinical trials targeting aging is the prolonged time between intervention and clinical outcomes (e.g., incidence of age-related morbidities) and surrogate endpoints are desperately needed. The first clinical trial aimed at delaying the processes that cause aging (TAME: Targeting Aging with Metformin) will soon begin (Barzilai et al. 2016 Cell Metab. 23:1060-1065). If this trial is successful, new clinical trials will quickly follow. For these studies, surrogate endpoints will dramatically improve the economy and timescale in which we can measure the effects of interventions on biological age (Niedernhofer et al. 2017 Ageing Res Rev. 35:241-249). Hundreds of studies have aimed to discover age-related changes in circulating factors including metabolites, advanced glycation end-products, exosome content, miRNA, and inflammatory molecules. However, these biomarkers described in the art are either not effective or inconvenient for use. For example, measuring expression of p16 in CD3+ peripheral blood mononuclear cells by qRT-PCR as reported in Liu et al. (Aging Cell, 8:439-448 (2009)) requires flow cytometry to isolate CD3+ cells.

Thus, there is an unmet need in the art for more reliable and easily detectable biomarkers that correlates with biological age, and related methods for determining biological age. The present document is directed to this and other unmet needs in the art.

### SUMMARY

In one aspect, this document provides methods for determining biological age or aging condition of a mammalian test subject (e.g., a human). The methods involve (a) determining circulating MCP-1 level in the test subject, and (b) comparing the determined MCP-1 level to average circulating MCP-1 level of control subjects of the same chronological age as that of the test subject. These allow determining aging condition of the test subject (e.g., aging well or aging poorly) or whether the biological age of the test subject is younger or older than the chronological age of the test subject. Some methods of the invention can further include generating or obtaining a standard scale of average circulating MCP-1 levels of control subjects of at least 2 different chronological age groups. In some of these embodiments, one of the at least 2 different chronological age groups is of the test subject's chronological age.

In some methods of this document, the average circulating MCP-1 level of healthy control subjects (e.g., healthy humans) is determined in the same manner as that used for determining circulating MCP-1 level in the test subject. In some methods, circulating MCP-1 level is determined with a blood sample from the test subject. In some of these embodiments, the blood sample is a peripheral plasma or serum sample. In some methods, the test subject and control subjects are humans. In some of these embodiments, the test subject and control subjects are free of or are not afflicted with inflammatory diseases.

In another aspect, this document provides methods for determining whether a medical treatment or intervention regimen for reversing or slowing down aging in a test subject (e.g., a human) is effective. The methods entail (a) determining at the beginning of the medical treatment or intervention regimen the test subject's biological age, (b) determining during the course and/or at the conclusion of the medical treatment or intervention regimen the test subject's biological age, and then (c) comparing the test subject's biological ages determined at the different measurement points. In the practice of the methods, the test subject's biological age is determined by measuring circulating MCP-1 level in the test subject and comparing the measured level to average circulating MCP-1 levels of control subjects of different chronological age groups. If there is a decline of the determined biological age, or a slower increase of the determined biological age relative to increase of the test subject's chronological age, it means an effectiveness of the medical treatment or intervention regimen in reversing or slowing down aging.

In another aspect, this document provides methods for identifying a mammal (e.g., a human) as being at risk for developing one or more adverse events (e.g., post-operative adverse events) following cardiovascular surgery. For example, a mammal scheduled for cardiovascular surgery can be assessed to determine the level of MCP-1 polypeptide expression within the mammal. If the mammal has an elevated level of MCP-1 polypeptide expression, then the mammal can be classified as being at risk of developing one or more adverse events such as sensitivity to anesthesia, poor wound healing, poor mechanical ventilation time, extended hospitalization, sundown syndrome, stroke, in hospital death, or a requirement for extended physical or occupational therapy. See, also, Rodrigues et al., Arq. Bras. Cardiol., 109:299-306 (2017).

Some methods for assessing treatment effectiveness can additionally include creating or obtaining a standard scale of average circulating MCP-1 levels of control subjects of at least 2 different chronological age groups. In some of these embodiments, one of the at least 2 different chronological age groups is of the test subject's chronological age. In some preferred embodiments, the average circulating MCP-1 levels of control subjects are determined in the same manner as that used for determining circulating MCP-1 level in the test subject. In various embodiments, circulating MCP-1 level is determined with a blood sample from the test subject and the control subjects. For example, the test can be performed with peripheral blood plasma or serum samples. In some preferred embodiments, the test subject for practicing the methods of the invention and the control subjects are humans. In some of these embodiments, the test subject and the control subjects are not suffering from or suspected of having inflammatory diseases.

In another aspect, this document features a method for slowing the progression of biological aging. The method comprises, or consists essentially of, administering a composition comprising a senolytic agent to a mammal identified as having an elevated level of circulating MCP-1 as compared to an average circulating MCP-1 level of healthy control mammals of the same chronological age as the mammal. The mammal can be a human. The human can have a chronological age that is over 55 years. The elevated level of circulating MCP-1 can be at least about 10 percent greater than the average circulating MCP-1 level.

In another aspect, this document features a method for slowing the progression of biological aging. The method comprises, or consists essentially of, administering a composition comprising a senolytic agent to a mammal identified as having a biological age based at least in part on an elevated level of circulating MCP-1, wherein the biological age is greater than the chronological age of the mammal. The mammal can be a human. The human can have a chronological age that is over 55 years. The biological age can be at least about 10 percent greater than the chronological age.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Circulating MCP-1 levels correlate with biological age. (A) Detection of MCP-1 in the serum of mice by ELISA. All mice were WT fl of varying ages and gender. (B) Linear regression analysis of the same data showing a highly significant correlation between serum MCP-1 and chronological age. (C) Graphing of the same date by gender. (D) MCP-1 serum concentrations were quantified by ELISA in progeroid Ercc1^{-/Δ} and Bubr1^{H/H} mice and WT littermate controls. (E) Genetic depletion of NF-κB in p65^{+/-};Ercc1^{-/Δ} mice, which extends the healthspan of the progeroid mice, reduces MCP-1 levels relative to Ercc1^{-/Δ} mice. 5-6 mice were used per group except for Bubr1 and their respective wild-type controls. (F) 16 week-old Ercc1^{-/Δ} mice (5-6 per group) treated with vehicle (Veh) or a combination of the senolytic drugs dasatinib and quercetin (D+Q) weekly starting at 4-6 weeks, and (G) 26 month-old WT mice (6 per group) that were placed on a rapamycin (Rapa) or control (Ctrl) diet for 8 weeks prior to analysis of serum MCP-1 by ELISA. Values represent the mean±SD, two tailed t test. p<0.05*, p<0.01**, p<0.001***, p<0.0001****, p<0.00001*****.

FIG. 2. Circulating MCP-1 levels are elevated in frail older adults. Plasma MCP-1 concentrations were quantified by a Luminex platform. Frail individuals possessed three or more of the following criteria: slow gait, weak grip, reduced physical activity, low endurance, and unintentional weight loss. Graphed are individual values, the mean±S.E.M. (Non-frail n=27, Frail n=36, Mann-Whitney test, **p=0.009).

FIG. 3. Multiplex ELISA of serum metabolic hormones and peptides. Serum from 2-3 month old and 24 month old WT mice were analyzed for 14 different hormones and peptides using the Milliplex Mouse Metabolic Hormone Panel Kit. 5-15 mice per group.

FIG. 4. The coefficient of variation is plotted for cytokines/chemokines measured in 78 females from the HANDLS cohort from the “CRP cohort”.

FIG. 5. Analysis of circulating MCP-1 in progeriod mice. MCP-1 was measured in the serum of age-matched WT and Ercc1^{-/Δ} mice using the Milliplex Mouse Metabolic Hormone Panel Kit. n=9-11 mice per group. The values represent the mean±SD. ****p<0.0001 using a two-tailed Student's t test.

FIG. 6. Fibroblasts derived from progeroid mice express elevated levels of Mcp1. (A) WT and Ercc1^{-/-} primary MEFs were analyzed for expression of Mcp1 at passage 2 (P2) and 7 (P7) by qPCR. (B) Conditioned media was analyzed for MCP-1 expression by ELISA. Senescence markers (C) p16 and (D) p21 expression in cell lysates was also measured by qPCR. Data was analyzed by the ΔΔCt method and expression was normalized to Gapdh expression. Values represent the mean±SD for n=3 independent MEF lines per group. A one-way ANOVA was used for statistical analysis, p<0.05*, p<0.01**, p<0.001***, p<0.0001****

FIG. 7. Representative images of kidney sections from old WT mice+/−rapamycin treatment. (A) Kidney section from a 26 month-old C57Bl/6 mouse treated with oral rapamycin for 8 weeks showed less severe age-related lesions, including mild glomerulonephropathy and mild lymphoid aggregates (circled), than a kidney section from a placebo treated mouse (B), which showed moderate glomerulonephropathy and moderate lymphoid aggregates (circled on the right), and an infarcted area (circled on the left).

FIG. 8. Analysis of age-related lesions in inbred and F1 hybrid mice. Total composite lesion scores for C57BL/6Jnia and C57BL/6Jnia:Balb/cBy mice (n=8) based on histopathologic analysis of liver, kidney, lungs, and heart. Values represent the mean±SD., p<0.05*, p<0.01**, using a two-tailed Student's t test.

FIG. 9. Stratification of MCP-1 concentration and frailty status by gender. Graphed are individual values, the mean±S.E.M. Mann-Whitney test.

FIG. 10. Circulating MCP-1 levels compared to post-operative adverse events. A trend of increased MCP-1 levels was observed in individuals who had at least one adverse event following cardiovascular surgery. Graphed are individual values, the mean±S.E.M. (No AE n=10, Any AE n=52, Mann-Whitney test).

FIG. 11. Circulating MCP-1 levels increase with chrological age. MCP-1 plasma concentrations were quantified by a Luminex platform in female and male Mayo Clinic Biobank participants. Graphed are individual values, the mean±S.E.M. (n=280, Spearman correlation).

FIG. 12. Circulating levels of MCP-1 were measured by ELISA. n=5 mice per group. One-way ANOVA with Tukey's multiple comparison test.

FIG. 13. Circulating levels of MCP-1 were measured in the serum of Vav-iCre^{+/-}; Ercc1^{-/fl} mice and sibling controls using the Luminex platform. n=6-10 mice per group. Values represent mean±SD. Two-tailed unpaired Student's t test. *p<0.05, **p<0.01.

FIG. 14. Circulating MCP-1 levels in recipient were measured 15 days post-injection. MCP-1 levels were significantly increased in recipient mice transplanted with “aged” immune cells. Values represent mean±SD. One-way ANOVA with Tukey's test. **p<0.01.

FIG. 15. Circulating level of MCP-1 were measure in progeroid Ercc1^{-/Δ} mice and age-matched WT mice. Values represent mean±SD. One-way ANOVA with Tukey's test. **p<0.01, ***p<0.001, ****p<0.0001.

FIG. 16. MCP-1 levels in the serum of 4-6 mo Ckmm-Cre^{+/-}; Ercc1f mice.

FIG. 17. MCP-1 levels in the serum of 6-9 month-old Rip-cre Ercc1^{-/fl}.

FIG. 18. MCP-1 levels in conditioned media.

### DETAILED DESCRIPTION

The present invention is directed to the use of circulating level of MCP-1 as a biomarker for biological age and related methods. MCP-1, also called CCL2 (chemokine C—C motif ligand 2) or small inducible cytokine A2, is a small protein found in serum of mammals. This 13 kD cytokine is secreted by senescent cells (Jin et al. 2016 Antioxid Redox Signal. 24:471-485) and functions to recruit monocytes, dendritic cells, macrophages and memory T cells to sites of injury or inflammation. Senescent cells and the pro-inflammatory cytokines that they secrete negatively affect tissue homeostasis and repair, leading to organ dysfunction and aging (van Deursen 2014). MCP-1 is implicated in the pathogenesis of many inflammatory diseases such as rheumatoid arthritis, atherosclerosis, and psoriasis.

Biological age is defined by the health or fitness of an individual, and lack of age-related diseases, irrespective of their chronological age (Liang et al. 2016 Int J Cardiol. 220:508-513). Biological age can be quite distinct from chronological age. For example, cancer survivors are biologically older than their chronological age due to exposure to genotoxic agents, while centenarians are frequently biologically younger than their chronological age (Ness et al. 2013 J Clin Oncol. 31:4496-4503; Govindaraju et al. 2015 Appl Transl Genom. 4:23-32). A biomarker of biological age in accessible bodily fluids or tissues would be extremely valuable for clinical trials testing anti-geronic factors, but also potentially for triaging patients facing onerous therapeutic procedures. As described herein, it was discovered in mice that circulating MCP-1 levels corresponded with biological rather than chronological age, and that they can respond to therapeutic interventions that alter normal aging. Additionally, it was found that circulating MCP-1 levels can also serve as a robust indicator of biological age in humans.

As detailed herein, the methods of the invention require quantifying the circulating level of MCP-1 in a mammalian subject and then comparing the measured level to a standard scale range of MCP-1 values based on control subjects of different chronological age. In some embodiments, the test subject (e.g., a human) to be examined with methods of the invention is one who is not afflicted or suspected of having any diseases or conditions that are associated with aberrant MCP-1 levels, e.g., inflammatory diseases noted above. Any blood sample (e.g., plasma, serum, or whole blood sample) from the subject can be employed in the practice of the invention. MCP-1 level in the blood sample can be readily determined in accordance with the protocols described herein or methods routinely practiced in the art. For example, ELISA kits that are commercially available (e.g., from Thermofisher or LifeSpan BioSciences) provide quick and accurate means for measuring circulating MCP-1 level in a test subject. Once the MCP-1 level of the test subject is determined, it is then compared with a standard scale of average MCP-1 level in healthy control subjects of varying chronological ages. Thus, in addition to measuring circulating MCP-1 in the test subject, the methods of the invention also require obtaining such a chronological age scale of MCP-1 levels, as explained below.

In some embodiments, the chronological age scale is obtained by collecting and compiling circulating MCP-1 levels from control subjects of different age groups that have been reported in the art. In some embodiments, the chronological age scale is obtained by measuring circulating MCP-1 levels in healthy control subjects of different age groups. In various embodiments, the control subjects for obtaining or creating the chronological age scale should include control subjects of at least 2, 3, 4, 5, 6, 7, 8, 9, 10 or more different age groups. Typically, one of the different age groups include control subjects who are of the same age as the chronological age of the test subject. The different age groups can be, e.g., 20, 25, 30, 35, 40, 45, 50, 55, 60, 65, 70, 75, 80, 85, 90, 95 and 100 years of age, including any age between these specified numbers. In order to arrive at an average MCP-1 level for each age group, each age group should preferably contain at least 2, 3, 4, 5, 6, 7, 8, 9, 10 or more subjects. Relative to the chronological age of the test subject, the chosen control subjects can include (1) those whose chronological ages are at least 5, 10, 15, 20 or more years younger, (2) those about the same age, and (3) those whose chronological ages are at least 5, 10, 15, 20 or more years older. Preferably, the same blood samples (e.g., peripheral blood or serum) are obtained from the control subjects as that obtained from the test subject. In addition, the same experimental protocols are used to measure MCP-1 levels from the control subjects as that employed from the test subject.

Upon comparing the circulating MCP-1 level of the test subject to the standard chronological age MCP-1 scale, aging condition or biological age of the test subject can then be determined. Thus, if the test subject's MCP-1 level is significantly or substantially higher than that of his or her chronological age peers, the test subject is considered biologically older or aging poorly. For example, if the MCP-1 level of a 45 year old test subject is about the same as that of control subjects in the 55 year old age group, the test subject is regarded as having a biological age of 55 years old. Similarly, if the test subject's MCP-1 level is significantly or substantially lower than that of his or her chronological age peers, the test subject is considered biologically younger or aging well. For example, if the MCP-1 level of a 45 year old test subject is about the same as that of control subjects in the 35 year old age group, the test subject is regarded as having a biological age of 35 years old.

In some cases, the methods and materials described herein can be used for predicting the risk of a subject (e.g., a human) to develop one or more adverse events (e.g., post-operative adverse events) following cardiovascular surgery. Examples of adverse events include, without limitation, post-operative adverse events, sensitivity to anesthesia, poor wound healing, poor mechanical ventilation time, extended hospitalization, sundown syndrome, stroke, in hospital death, or a requirement for extended physical or occupational therapy. See, also, Rodrigues et al., Arq. Bras. Cardiol., 109:299-306 (2017).

Other examples of adverse events include, without limitation, adverse events drug toxicity, surgical complications including myocardial infarction, new arrhythmia, new conduction abnormality, stroke, deep vein thrombosis, pulmonary emboli, pneumonia, pleural effusion, renal insufficiency, seizure disorder, hypotension, tachycardia, bradycardia, urinary tract infection, other infections, and/or acute dementia. Examples of cardiovascular surgeries include, without limitation, valve replacement surgery and coronary artery bypass. For example, the level of MCP-1 in a subject can be used to identify a mammal (e.g., a human) at risk for developing one or more adverse events following a cardiovascular surgery such as valve replacement surgery (e.g., for severe aortic stenosis). In some cases, increased levels of MCP-1 in a subject can be used to predict that the subject is at risk of developing one or more adverse events following cardiovascular surgery.

In addition to determining one's aging condition or biological age, the methods of the invention can also be employed for assessing effectiveness, monitoring progresses or quantifying results of medical treatments or therapeutic interventions, as well as other regimen including physical or mental activities, in reversing or slowing down aging of a test subject. This can be readily accomplished by performing the test noted above prior to, during the period of, and subsequent to receiving the treatment or intervention by the subject. If the MCP-1 level of the subject relative to his age peers decreases during or after the treatment or intervention, it can be concluded that the treatment or intervention is effective in reversing or slowing down aging. As the treatment or intervention can last for years, the determined MCP-1 level needs to be compared to average circulating MCP-1 level of control subjects with the test subject's chronological age at each specific measurement point in mind. To phrase it differently, these methods can entail determining the test subject's biological age prior to, during the course of, and/or at the conclusion of the treatment or intervention. At each measurement point, the test subject's biological age is determined by measuring circulating MCP-1 level in the test subject and comparing the measured level to average circulating MCP-1 levels of control subjects. This is achieved by obtaining a standard scale of circulating MCP-1 levels of control subjects of different chronological age groups. For example, the scale of circulating MCP-1 levels can include average circulating MCP-1 levels of control subjects of at least 2 different chronological age groups. In some embodiments, one of the different chronological age groups used for generating the standard scale is of the test subject's chronological age at the time of the measurement. If the test subject's biological age declines over the course of the treatment or intervention, it means the treatment or intervention is effective in reversing aging. Alternatively, if the increase of the test subject's biological age during the course of the treatment or intervention is slower relative to increase of the test subject's chronological age, it means the medical treatment or intervention regimen is effective in slowing down aging.

The invention can employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Sambrook et al, ed. (1989) Molecular Cloning A Laboratory Manual (2nd ed.; Cold Spring Harbor Laboratory Press); Sambrook et al, ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al, eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000).

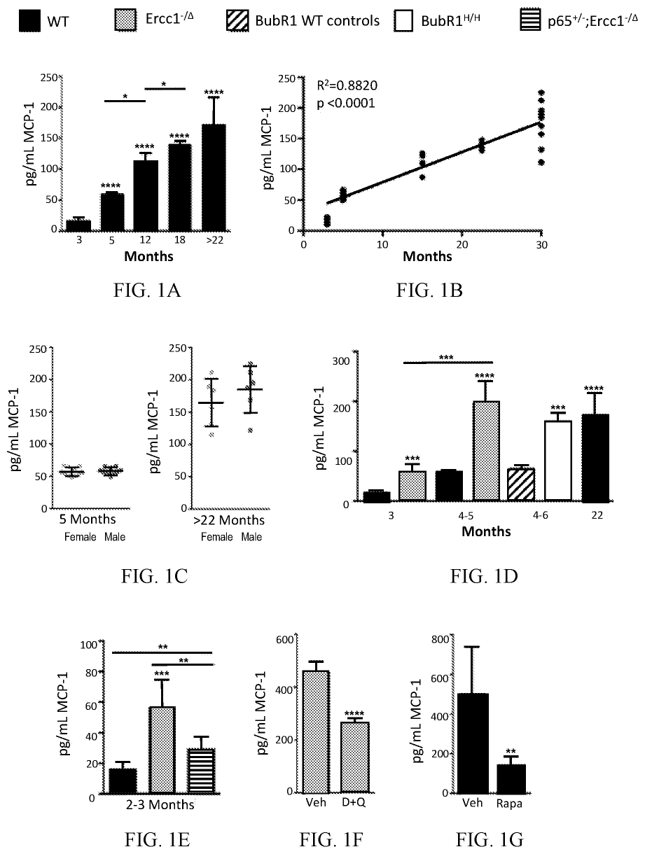

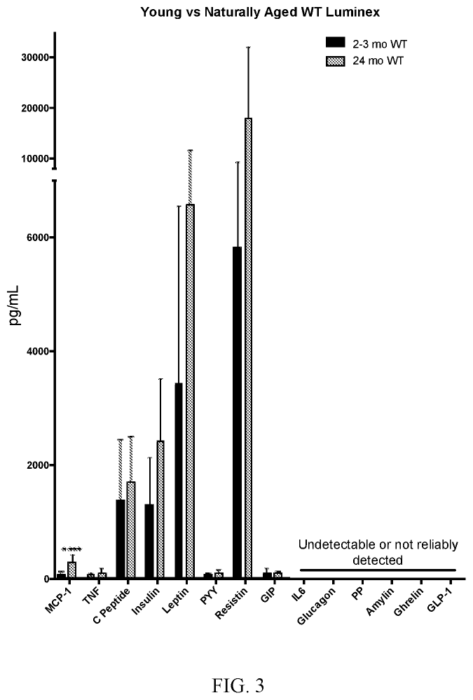

As demonstrated herein, these methods of the invention can be readily applied in determining biological age of mammalian subjects, e.g., human subjects. As specific exemplifications, multiple serum cytokines and chemokines were measured in young and old WT mice using a Luminex platform designed to detect 14 circulating peptides in mouse plasma (FIG. 3). Notably, neither TNFα nor IL-6 were increased in aged mice compared to young, which was confirmed by ELISA. In contrast, in this targeted analysis, MCP-1 was the only peptide that increased significantly and reproducibly with chronological age (FIG. 1A). Monocyte chemoattractant protein-1 (MCP-1/CCL2) is a chemokine produced by a number of cell types including endothelial, epithelial, mesangial, myocytes, monocytes and microglial cells, either in a constitutive manner or in response to various stimulants, such as oxidative stress, cytokines and growth factors (Deshmane et al. 2009). MCP-1 is a potent monocyte chemoattractant that binds the CCR2 receptor and induces monocytes to exit the bloodstream to become tissue macrophages in response to inflammatory signals (Deshmane et al. 2009).

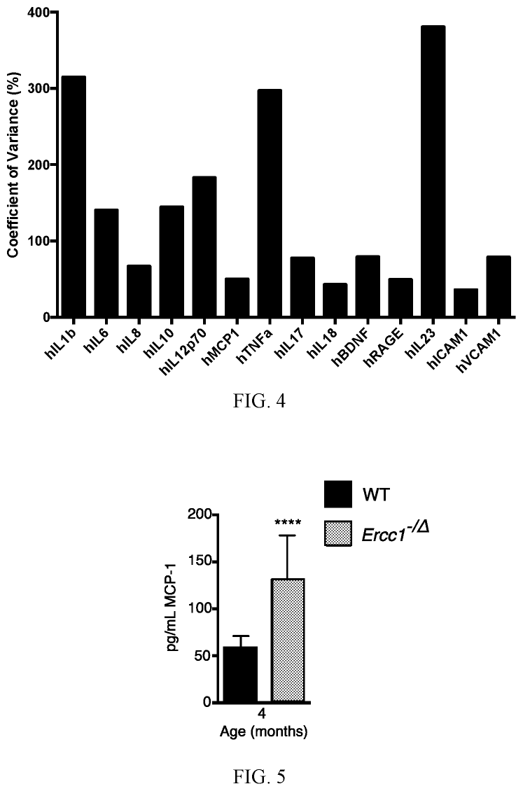

Numerous studies previously demonstrated that plasma levels of MCP-1 correlate with chronologic age in humans (Inadera et al. 1999 J Interferon Cytokine Res. 19:1179-1182; Deo et al. 2004 J Am Coll Cardiol. 44:1812-1818; Mansfield et al. 2012 Clin Exp Immunol. 170:186-193; Pinke et al. 2013 Immun Ageing. 10:22; Brouwers et al. 2015 Aging (Albany N.Y.). 7:319-333; Scully et al. 2016 J Infect Dis. 213:771-775) and mice (Chiao et al. 2011 Circ Cardiovasc Genet. 4:455-462). MCP-1 is a senescence-associated secretory phenotype (SASP) factor secreted by senescent cells (Jin et al. 2016 Antioxid Redox Signal. 24:471-485). SASP can promote secondary senescence in healthy cells (Coppe et al. 2010 Annu Rev Pathol. 5:99-118) and senescent cells have been demonstrated to promote aging and age-related disease (Baker et al. 2011 Nature. 479:232-236; Zhu et al. 2015 Aging cell. 14:644-658; Baker et al. 2016 Nature. 530:184-189). Circulating levels of MCP-1 are increased in patients with renal disease (Akdogan et al. 2015 Ren Fail. 37:1297-1302), cognitive impairment and Alzheimer's disease (Bettcher et al. 2016 Alzheimers Dement (Amst). 3: 91-97), atherosclerosis and cardiovascular disease (Deo et al. 2004 J Am Coll Cardiol. 44:1812-1818). MCP-1 is considered to be a marker of “inflammaging”, defined as chronic sterile inflammation that is associated with numerous age-related diseases (Franceschi & Campisi 2014 J Gerontol A Biol Sci Med Sci. 69 Suppl 1:54-9). Therefore, we focused on MCP-1 as a potential biomarker of biological age because it is readily measured in humans, with a relatively small coefficient of variation compared to other inflammatory markers (FIG. 4), and there is a rationale for it potentially correlating with aging rather than merely inflammation.

As previous shown in inbred C57BL/6 mice (Chiao et al. 2011 Circ Cardiovasc Genet. 4:455-462), MCP-1 levels increased linearly with the chronological age of WT fl mice (FVB/n;C57BL/6; FIG. 1B). It is interesting to note that the inter-individual variation in MCP-1 levels increased dramatically in older mice (FIG. 1A-B). This is consistent with aging being incredibly heterogeneous at the physiological and molecular level (Burd et al. 2013 Cell. 152:340-351; Lowsky et al. 2014 J Gerontol A Biol Sci Med Sci. 69:640-649). Also of note, no sex-based difference in MCP-1 levels were detected in mice (FIG. 1C).

To determine if MCP-1 levels corresponded with biological rather than chronological age, we measured serum MCP-1 in two unrelated models of accelerated aging. Ercc1^{-/Δ} mice model a human progeroid syndrome caused by defective DNA repair (Niedernhofer et al. 2006 Nature. 444:1038-1043), have a median lifespan of 5 months (Dolle et al. 2011), and spontaneously develop numerous diseases and pathologies associated with old age in humans (Table 1). BubR1^{H/H} mice age rapidly due to defective mitotic spindle assembly checkpoint and have a median lifespan of 6 months (Table 2). In both progeroid strains, serum MCP-1 levels were significantly increased compared to age-matched WT mice (FIG. 1D). To validate these ELISA data, we used Luminex to measure MCP-1 in Ercc1^{-/Δ} mouse serum and observed a significant increase in MCP-1 compared to age-matched controls (FIG. 5). Notably, at an age equivalent to the median lifespan of Ercc1^{-/Δ} and BubR1^{H/H} mice, serum MCP-1 levels were equivalent to that of 22 month-old WT mice, an age when WT mice begin to display age-related pathologies (Fox 2007). The data are not strain-dependent as the Ercc1^{-/Δ} and naturally aged mice were in an fl (C57BL/6;FVB) genetic background, while the BubR1^{H/H} mice were C57BL/6.

TABLE 1
Universal changes that occur with aging
in humans (Health & Medicine 2007).

| Human age-related | | Ercc1^{-/Δ} mouse | |
|---|---|---|---|
| changes | | age-related changes | References |
| 1. | Brain/ | cognitive decline | (Borgesius et al. 2011; |
| | memory | cerebral atrophy | Dolle et al. 2011; |
| | | brain vacuolization | Harkema et al. 2016) |
| 2. | Bones & | osteoporosis | (Vo et al. 2010; Chen |
| | joints | disc degeneration | et al. 2013) |
| 3. | Eyes & ears | loss of vision | (Spoor et al. 2012) |
| | | loss of hearing | |
| | | cataracts | |
| 4. | Digestive & | metabolic shift | (Karakasilioti et al. |
| | metabolic | | 2013) |
| 5. | Urogenital | renal tubule | (Dolle et al. 2011; |
| | | degeneration | Tilstra et al. 2012) |
| | | urinary incontinence | |
| | | testis atrophy | |
| 6. | Dental | ? | |
| 7. | Skin | epidermal atrophy | (Weeda et al. 1997; |
| | | alopecia | Harkema et al. 2016) |
| | | greying | |
| | | loss of subcutaneous | |
| | | fat | |
| 8. | Function | muscle wasting | (Gregg et al. 2011; |
| | | ataxia | Tilstra et al. 2012) |
| | | falls | |

Borgesius N Z, de Waard M C, van der Pluijm I, Omrani A, Zondag G C, van der Horst G T, Melton D W, Hoeijmakers J H, Jaarsma D, Elgersma Y (2011). Accelerated age-related cognitive decline and neurodegeneration, caused by deficient DNA repair. J Neurosci. 31, 12543-12553.
Chen Q, Liu K, Robinson A R, Clauson C L, Blair H C, Robbins P D, Niedemhofer L J, Ouyang H (2013). DNA damage drives accelerated bone aging via an NF-kappaB-dependent mechanism. J Bone Miner Res. 28, 1214-1228.
Dolle M E, Kuiper R V, Roodbergen M, Robinson J, de Vlugt S, Wijnhoven S W, Beems R B, de la Fonteyne L, de With P, van der Pluijm I, Niedernhofer L J, Hasty P, Vijg J, Hoeijmakers J H, van Steeg H (2011). Broad segmental progeroid changes in short-lived Ercc1(−/Delta7) mice. Pathobiol Aging Age Relat Dis. 1.
Gregg S Q, Robinson A R, Niedernhofer L J (2011). Physiological consequences of defects in ERCC1-XPF DNA repair endonuclease. DNA Repair (Amst). 10, 781-791.
Harkema L, Youssef S A, de Bruin A (2016). Pathology of Mouse Models of Accelerated Aging. Vet Pathol. 53, 366-389.
Health NIo, Medicine FotNLo (2007). NIH Medline Plus. (aoa-r change, ed\hat{ }eds): Friends of the National Library of Medicine, pp. 10-13.
Karakasilioti I, Kamileri I, Chatzinikolaou G, Kosteas T, Vergadi E, Robinson A R, Tsamardinos I, Rozgaja T A, Siakouli S, Tsatsanis C, Niedernhofer L J, Garinis G A (2013). DNA damage triggers a chronic autoinflammatory response, leading to fat depletion in NER progeria. Cell Metab. 18, 403-415.
Spoor M, Nagtegaal A P, Ridwan Y, Borgesius N Z, van Alphen B, van der Pluijm I, Hoeijmakers J H, Frens M A, Borst J G (2012). Accelerated loss of hearing and vision in the DNA-repair deficient Ercc1(delta/−) mouse. Mech Ageing Dev. 133, 59-67.
Tilstra J S, Robinson A R, Wang J, Gregg S Q, Clauson C L, Reay D P, Nasto L A, St Croix C M, Usas A, Vo N, Huard J, Clemens P R, Stolz D B, Guttridge D C, Watkins S C, Garinis G A, Wang Y, Niedernhofer L J, Robbins P D (2012). NF-kappaB inhibition delays DNA damage-induced senescence and aging in mice. J Clin Invest. 122, 2601-2612.
Vo N, Seo H Y, Robinson A, Sowa G, Bentley D, Taylor L, Studer R, Usas A, Huard J, Alber S, Watkins S C, Lee J, Coehlo P, Wang D, Loppini M, Robbins P D, Niedernhofer L J, Kang J (2010). Accelerated aging of intervertebral discs in a mouse model of progeria. J Orthop Res. 28, 1600-1607.
Weeda G, Donker I, de Wit J, Morreau H, Janssens R, Vissers C J, Nigg A, van Steeg H, Bootsma D, Hoeijmakers J H (1997). Disruption of mouse ERCC1 results in a novel repair syndrome with growth failure, nuclear abnormalities and senescence. Curr Biol. 7, 427-439.

TABLE 2
Comparison of mouse models of progeria.

| | Ercc1^{-/Δ} | Bubr1^{H/H} |
|---|---|---|
| Mutated gene | Ercc1 | Bubr1 |
| Consequence of | Compromised in | Defective mitotic |
| mutation | multiple DNA | spindle assembly |
| | repair pathways | checkpoint |
| % normal protein | <10% | 10% |
| expression | | |
| Organ systems affected: | | |
| Musculoskeletal | + | + |
| Dermatologic | + | + |
| Connective tissue | + | + |
| (adipose) | | |
| Neurologic | + | + |
| Hematologic | + | − |
| Sensorineural | + | − |
| Immunologic | + | − |
| Cardiovascular | + | + |
| Other | Renal dysfunction, | Impaired wound |
| | disc degeneration, | healing, cachexia, |
| | infertility | facial dimorphisms, |
| | | infertility |
| Lifespan | Median: 4 months; | Median: 6 months |
| | Maximum: 7.5 months | |
| Premature cell | + | + |
| senescence | | |

+/− indicates the presence of absence of involvement

To determine if MCP-1 levels can detect reduced biological age, serum chemokine levels were measured in p65^{+/-};Ercc1^{-/Δ} mice. We previously established that genetic depletion of the RelA/p65 subunit of NF-κB significantly extends the healthspan of Ercc1^{-/Δ} mice (Tilstra et al. 2012 J Clin Invest. 122:2601-2612). Indeed, p65+/−;Ercc1^{-/Δ} mice had significantly reduced circulating levels of MCP-1 compared to age-matched Ercc1^{-/Δ} mice (FIG. 1E). Together, these data support the conclusion that MCP-1 is a better marker of biological than chronological age.

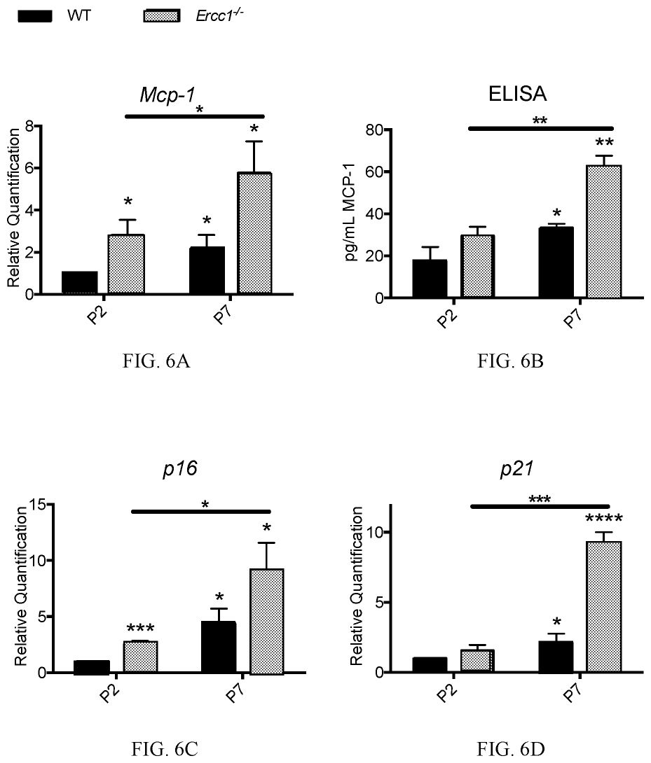

Mcp1 expression is increased in fibroblasts from Hutchinson-Gilford Progeria Syndrome patients compared to control cell lines (Csoka et al. 2004 Aging cell. 3:235-243). This was recapitulated in mouse embryonic fibroblasts derived from Ercc1-deficient mice. Mcp1 expression was elevated in Ercc1^{-/-} MEFs compared to WT as early as passage 2 and levels increased significantly in both WT and Ercc1^{-/-} cells with passaging (FIG. 6A). Similarly, MCP-1 protein abundance was higher in the media of p7 cells compared to p2, and significantly greater in Ercc1^{-/-} MEFs compared to WT (FIG. 6B). The MCP-1 data corresponded with a significant increase in the expression of other markers of cellular senescence in the Ercc1^{-/-} cells relative to WT (p16 and p21; FIG. 6C-D). Thus MCP-1 expression, at both the RNA and protein level, may serve as an indicator of the burden of senescent cells, which drive aging.

By definition, a biomarker of biological age should respond to therapeutic interventions proven to significantly improve healthspan or lifespan. Here, we measured serum MCP-1 in two distinct, established intervention paradigms. Genetic or pharmacologic ablation of senescent cells extends healthspan of mice (Zhu et al. 2015 Aging cell. 14:644-658; Baker et al. 2016 Nature. 530:184-189). A combination of two senolytic drugs (Dasatinib and quercetin) extends the healthspan of Ercc1^{-/Δ} mice and delays multiple age-related pathologies (Zhu et al. 2015 Aging cell. 14:644-658). In that study, Ercc1^{-/Δ} mice were treated weekly with a combination of Dasatanib (5 mg/kg) and quercetin (50 mg/kg) for 10 weeks, starting at 6 weeks of age. Here, we analyzed serum from these mice for circulating levels of MCP-1. Ercc1^{-/Δ} mice treated with D+Q had significantly lower circulating concentrations of MCP-1 than vehicle-treated controls (FIG. 1F). Of note, serum MCP-1 levels in the vehicle only group of Ercc1^{-/Δ} mice in this study is higher than that of untreated animals Ercc1^{-/Δ} mice (4-6 mth Ercc1^{-/Δ} mice in FIG. 1D was ˜175 pg/mL vs. ˜400 pg/ml in 4 mth-old mice in FIG. 1F). We attribute this to the repeated i.p. injections and frequent handling of the Ercc1^{-/Δ} mice in the latter study, which exacerbates their frailty.

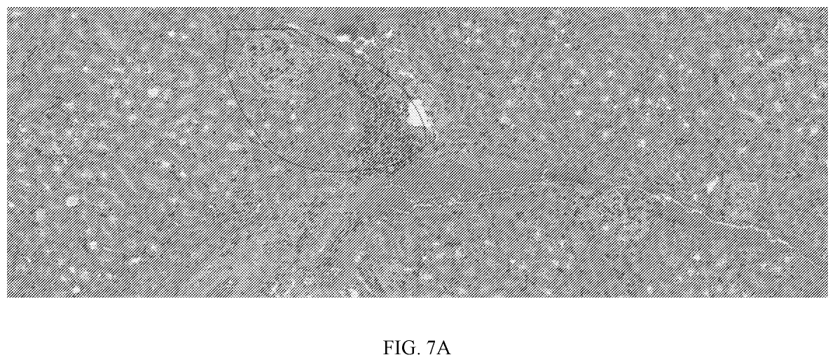

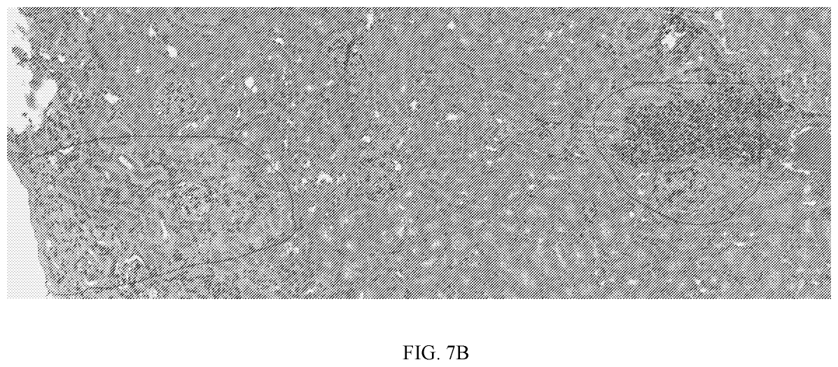

Rapamycin, an inhibitor of the mTOR kinase, causes a significant extension in the lifespan of WT mice (Harrison et al. 2009 Nature. 460:392-395). Furthermore, late-life intervention with rapamycin is sufficient to reduce multiple characteristics of cardiac aging (Dai et al. 2014). Two year-old C57BL/6J mice were fed a diet containing rapamycin (14 ppm for females or 42 ppm for males) or a control diet for two months. Longitudinal echocardiography demonstrated that rapamycin significantly reversed aging-related decline in cardiac performance and substantially attenuated cardiac hypertrophy, as previously described (Dai et al. 2014). In addition, rapamycin attenuated composite lesion scores in kidneys (FIG. 7), liver, and lungs of these mice by an average of 40, 41, and 29 percent, respectively. Composite lesion scores generated by a geropathology grading platform have been shown to increase in mice in an age-dependent manner and align with biological age (Ladiges et al. 2017 J Gerontol A Biol Sci Med Sci. 72:760-762). Serum levels of MCP-1 were significantly decreased in 26 month-old WT mice after treatment with rapamycin compared to controls (FIG. 1G). These data provide strong experimental evidence that in pre-clinical models, circulating MCP-1 levels serve as a surrogate endpoint, i.e., it responds to interventions that improve clinical endpoints of healthy aging, irrespective of the chronological age of the animals.

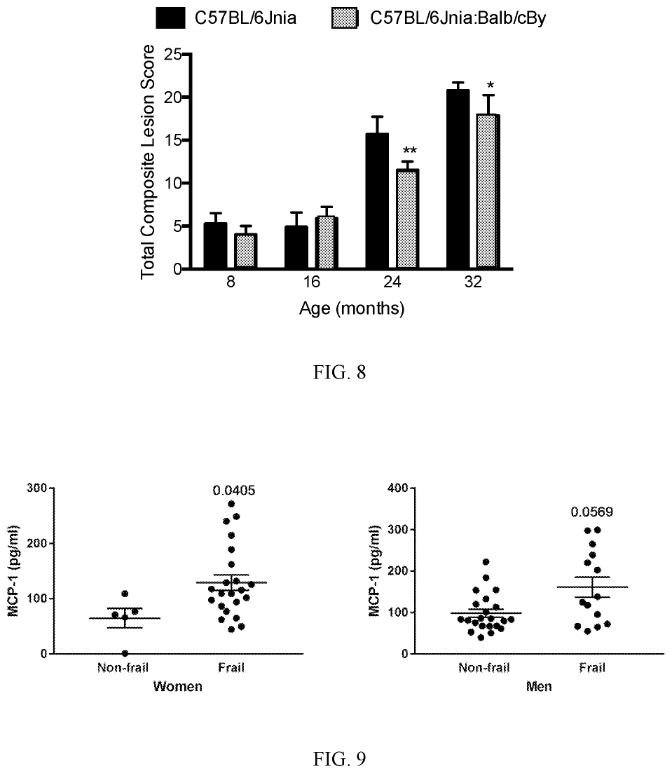

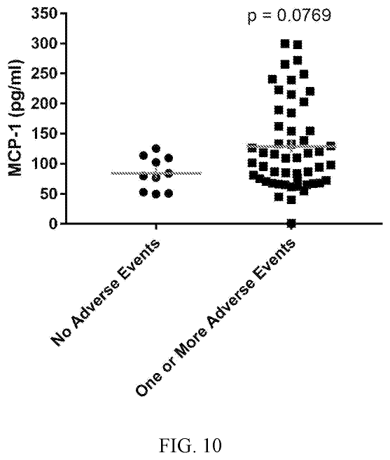

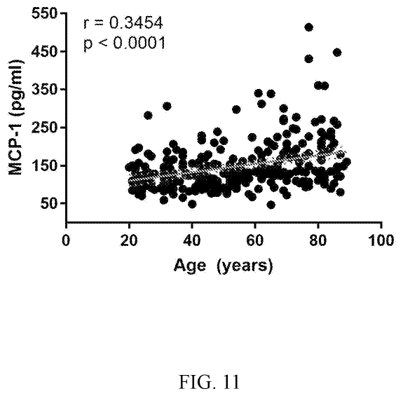

Interestingly, MCP-1 levels were greater in inbred C57BL/6NJ mice compared to age-matched fl mice (˜500 pg/ml for vehicle-treated 26 month-old C57BL/6NJ mice in FIG. 1F compared to ˜175 pg/ml for fl C57BL/6J:FVB/NJ mice >22 months of age in FIG. 1A). This suggests that fl mice are biologically younger than chronologically age-matched inbred mice. In fact, fl mice are healthier and longer-lived than inbred mice (Flurkey et al. 2006 Mouse models in aging research. In The Mouse in Biomedical Research: Normative Biology, Husbandry, and Models. (Fox et al., eds). Burlington, Mass.: Academic Press, pp. 637-672). In addition, inbred mice accumulate numerous age-related histopathological lesions in multiple organs at an earlier age than fl mice (Ladiges et al. 2017 J Gerontol A Biol Sci Med Sci. 72:760-762) (FIG. 8). The fact that rapamycin lowers serum MCP-1 levels to a range consistent with fl mice suggests that rapamycin reverses aging.

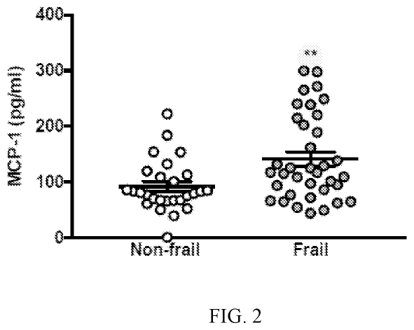

Our findings demonstrate striking associations between circulating MCP-1 concentrations and biological age in multiple mouse strains. However, establishing whether a comparable relationship exists in humans is necessary for determining translational utility. Accordingly, we measured plasma MCP-1 levels in a cohort of older adults undergoing valve replacement surgery for severe aortic stenosis (Table 3). Cardiovascular health study (CHS) frailty testing was conducted as a surrogate measure of biological age, using the presence of three or more frailty criteria (slow gait, weak grip, reduced physical activity, low endurance, and unintentional weight loss) as an operational frailty definition (Fried et al. 2001 J Gerontol A Biol Sci Med Sci. 56(3):M146-56). Within this sample of 27 women and 36 men, mean age of 81 years, circulating MCP-1 levels were 54% higher in frail participants (FIG. 2). Since frailty status was associated with age and sex (Table 3), we also applied linear regression analyses to control for these factors. A one unit increase in the natural log of MCP-1 levels was associated with a 0.86 unit increase in frailty score, and the strength and significance of this relationship did not meaningfully change after adjusting for age, sex, or combined age and sex (Table 4). To further explore potential sex differences, we split our sample into male and female groups and applied univariate linear regression. A one unit increase in natural log MCP-1 levels corresponded to a 0.47 and 1.45 unit increase in frailty score in women (p=0.004) and men (p=0.002), respectively (Table 5; FIG. 9). Thus, we conclude that circulating MCP-1 concentrations are a robust indicator of biological age in humans, regardless of sex.

TABLE 3
Study sample demographic characteristics
stratified by frailty status.

| | Non-Frail (n = 27) | | Frail Subjects (n = 36) | | z |
|---|---|---|---|---|---|
| | Mean (SD) or Number (%) | | | | p-value |
| Age (yrs) | 79.0 | (8.3) | 82.9 | (5.8) | 0.046¹ |
| Male | 22 | (82%) | 14 | (39%) | <0.001² |
| BMI | 28.8 | (2.8) | 31.0 | (6.8) | 0.40³ |
| Weight (kg) | 85.5 | (13.8) | 82.2 | (19.3) | 0.45¹ |
| Height (cm) | 172.0 | (10.7) | 162.8 | (9.8) | 0.001¹ |

¹Unpaired t-test,
²Chi-square,
³Mann-Whitney

TABLE 4
Linear regression relationships between MCP-1 and frailty
score, adjusted for the indicated covariates.

| Predictor Variables | β | Std. Error | 95% CI | p-value |
|---|---|---|---|---|
| MCP-1 | 0.86 | 0.25 | 0.36-1.35 | 0.001 |
| MCP-1 + Age | 0.90 | 0.23 | 0.43-1.36 | <0.001 |
| MCP-1 + Sex | 0.94 | 0.22 | 0.50-1.38 | <0.001 |
| MCP-1 + Age + Sex | 0.96 | 0.21 | 0.53-1.39 | <0.001 |

*MCP-1 values were natural log transformed to fit a more normal distribution.

TABLE 5
Linear regression relationship between MCP-1
and frailty score among women and men.

| Predictor Variables | β | Std. Error | 95% CI | p-value |
|---|---|---|---|---|
| MCP-1 in Women | 0.74 | 0.24 | 0.26-1.23 | 0.004 |
| MCP-1 in Men | 1.45 | 0.43 | 0.57-2.33 | 0.002 |

*MCP-1 values were natural log transformed to fit a more normal distribution.

This document also provides methods and materials for treating aging or slowing the progression of aging in a mammal identified as having (a) an elevated level of MCP-1 polypeptide expression as described herein or (b) a biological age that is greater than that mammal's chronological age based on the level of MCP-1 polypeptide expression as described herein. For example, a mammal (e.g., a human) that was identified to have a biological age that is greater than that mammal's chronological age based on (or based, at least in part, on) the level of MCP-1 polypeptide expression as described herein can be administered a composition containing one or more senotherapeutic agents to slow that mammal's biological aging.

A composition containing one or more senotherapeutic agents can include any appropriate senotherapeutic agent(s). In some cases, a senotherapeutic agent can be a senolytic agent (i.e., an agent having the ability to induce cell death in senescent cells). Examples of senolytic agents that can be used as described herein (e.g., to slow the progression of biological aging in a mammal identified as described herein) can include, without limitation, dasatinib, quercetin, navitoclax, A1331852, A1155463, ABT-737, fisetin, luteolin, geldanamycin or other HSP90 inhibitors, piperlongumine, panobinostat, FOX04 peptides, and nutlin3a. In some cases, a senotherapeutic agent can be a senomorphic agent (i.e., an agent having the ability to suppress senescent phenotypes without cell killing). Examples of senomorphic agents that can be used as described herein (e.g., to slow the progression of biological aging in a mammal identified as described herein) can include, without limitation, ruxolitinib, metformin, and rapamycin. In some cases, a senotherapeutic agent used as described herein can be an orally-active senotherapeutic agent. A senotherapeutic agent can be any appropriate type of molecule. For example, a senotherapeutic agent can be a small molecule. In some cases, one, two, three, four, five or more different senotherapeutic agents can be used in combination or sequentially to slow the progression of biological aging in a mammal identified as described herein.

When treating a mammal to slow the progression of biological aging in the mammal identified as described herein, the mammal can be any appropriate mammal. In some cases, a mammal can be an older mammal (e.g., a human over 55 years of age). Examples of mammals that can be treated using a composition containing one or more senotherapeutic agents as described herein include, without limitation, humans, non-human primates such as monkeys, dogs, cats, horses, cows, pigs, sheep, mice, rats, hamsters, guinea pigs, and goats.

In some cases, a composition containing one or more senotherapeutic agents can be formulated into a pharmaceutically acceptable composition for administration to a mammal that was identified to have (a) an elevated level of MCP-1 polypeptide expression as described herein or (b) a biological age that is greater than that mammal's chronological age based on (or based, at least in part, on) the level of MCP-1 polypeptide expression as described herein. For example, one or more senotherapeutic agents can be formulated together with one or more pharmaceutically acceptable carriers (additives) and/or diluents. Pharmaceutically acceptable carriers, fillers, and vehicles that can be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol (PEG; e.g., PEG400), sodium carboxymethylcellulose, polyacrylates, waxes, polyethylene-polyoxypropylene-block polymers, and wool fat.

In some cases, when a composition containing one or more senotherapeutic agents is administered to a mammal (e.g., a human) that was identified to have (a) an elevated level of MCP-1 polypeptide expression as described herein or (b) a biological age that is greater than that mammal's chronological age based on (or based, at least in part, on) the level of MCP-1 polypeptide expression as described herein, the composition can be designed for oral or parenteral (including subcutaneous, intramuscular, intravenous, and intradermal) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition containing one or more senotherapeutic agents can be formulated for oral administration.

A composition containing one or more senotherapeutic agents can be administered to a mammal that was identified to have (a) an elevated level of MCP-1 polypeptide expression as described herein or (b) a biological age that is greater than that mammal's chronological age based on (or based, at least in part, on) the level of MCP-1 polypeptide expression as described herein in any appropriate dose. Effective doses can vary depending on the route of administration, the chronological age and general health condition of the subject, excipient usage, the possibility of co-usage with other therapeutic treatments such as use of other agents, and the judgment of the treating physician. An effective amount of a composition containing one or more senotherapeutic agents can be any amount that slows the progression of biological aging without producing significant toxicity to the mammal. For example, an effective amount of dasatinib (D) can be from about 1 milligrams per kilogram body weight (mg/kg) to about 20 mg/kg (e.g., about 5 mg/kg). For example, an effective amount of quercetin (Q) can be from about 10 mg/kg to about 200 mg/kg (e.g., about 50 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition being treated may require an increase or decrease in the actual effective amount administered.

A composition containing one or more senotherapeutic agents can be administered to a mammal that was identified to have (a) an elevated level of MCP-1 polypeptide expression as described herein or (b) a biological age that is greater than that mammal's chronological age based on (or based, at least in part, on) the level of MCP-1 polypeptide expression as described herein in any appropriate frequency. The frequency of administration can be any frequency that slows the progression of biological aging without producing significant toxicity to the mammal. For example, the frequency of administration can be from about once a day to about once a month, from about three times a day to about once a week, or from about every other day to about twice a month. In some cases, a composition containing one or more senotherapeutic agents can be administered for three consecutive days every two weeks. The frequency of administration can remain constant or can be variable during the duration of treatment. As with the effective amount, various factors can influence the actual frequency of administration used for a particular application. For example, the effective amount, duration of treatment, use of multiple treatment agents, and route of administration may require an increase or decrease in administration frequency.

A composition containing one or more senotherapeutic agents can be administered to a mammal that was identified to have (a) an elevated level of MCP-1 polypeptide expression as described herein or (b) a biological age that is greater than that mammal's chronological age based on (or based, at least in part, on) the level of MCP-1 polypeptide expression as described herein for any appropriate duration. An effective duration for administering a composition containing one or more senotherapeutic agents can be any duration that slows the progression of biological aging without producing significant toxicity to the mammal. For example, the effective duration can vary from several days to several months or years to a lifetime. In some cases, the effective duration can range in duration from about 10 years to about a lifetime. When contacting a graft with a composition provided herein, the effective duration can be from about 30 minutes to 2 days. Multiple factors can influence the actual effective duration used for a particular treatment. For example, an effective duration can vary with the frequency of administration, effective amount, use of multiple treatment agents, and route of administration.

In certain instances, a course of treatment can be monitored. Any appropriate method can be used to monitor biological aging. For example, MCP-1 polypeptide expression levels can be assessed using any appropriate methods and/or techniques and can be assessed at different time points as described herein.

The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

The following examples are provided to further illustrate the invention but not to limit its scope. Other variants of the invention will be readily apparent to one of ordinary skill in the art and are encompassed by the appended claims.

### Example 1

### Experimental Procedures:

Mice.

Wild-type, Ercc1^{-/Δ} and p65^{+/-};Ercc1^{-/Δ} mice for this study were in an fl C57B/6J and FVB background. Ercc1^{-/Δ} mice were generated by crossing Ercc1^{+/+} mice in a C57Bl/6J with Ercc1^{-/Δ} mice in a FVB/N genetic background. The fl background reduces strain-specific pathology while still allowing for analysis of genetically identical animals. Mice were given a unique identifier by ear punch. Genomic DNA was isolated from ear tissue and the genotype of each animal was determined by Transnetyx (Cordova, Tenn.) or as described elsewhere (see, e.g., Ahmad et al., 2008 Mol Cell Biol. 28(16):5082-92). Wild-type (WT) fl littermates were used as young normal controls. The Bubr1^{H/H} mice and their WT controls were in a C57Bl/6 background as described elsewhere (see, e.g., Baker et al., 2004 Nat Genet. 36(7):744-9). Serum was isolated from animals at the time of euthanasia (using CO₂) by cardiac puncture.

Generation and Culture Conditions of Primary Mouse Embryonic Fibroblasts (MEFs).

Ercc1^{-/-} primary MEFs were prepared from day 13 embryos derived from crossing inbred C57BL/6 mice heterozygous for an Ercc1 null allele, as described elsewhere (see, e.g., Ahmad et al., 2008 Mol Cell Biol. 28(16):5082-92). Cell lines simultaneously derived from wild-type (WT) littermate embryos were used as controls. Primary MEFs were cultured in a 1:1 mixture of Dulbecco's modified Eagle's medium and Ham's F10 with 10% fetal bovine serum, non-essential amino acids and antibiotics, and incubated at 3% 02. Three independent MEF lines of each genotype were used.

RNA Isolation and qPCR.

Total RNA was isolated from MEFs using RNeasy isolation kit (Qiagen, Valencia, Calif.). Total RNA was quantified using aNanodrop spectrophotometer (Thermo Fisher, Waltham, Mass.) and 1 μg of total RNA was used to generate cDNA with the Transcriptor First Strand cDNA synthesis kit (Roche, Basel Switzerland) according to the manufacturer's specification. Gene expression changes in Mcp1, p16 and p21 were quantified by qPCR reactions using 20 μL reaction volumes using a StepOne thermocycler (Thermo Fisher, Waltham, Mass.) with input of 50 ng (Gapdh, p21, Mcp1) or 100 ng (p16) total RNA per reaction. Reactions were performed in duplicate for three separate experiments. Data was analyzed by ΔΔCt method and expression was normalized to Gapdh. qPCR primer sequences are listed in Table 6.

TABLE 6
qPCR primers.

| Primer | Sequence (SEQ ID NO:) |
|---|---|
| Cdkn1a (p21) Fwd | 5′-GCCTTAGCCCTCACTCTGTG-3′ (1) |
| Cdkn1a (p21) Rev | 5′-AGCTGGCCTTAGAGGTGACA-3′ (2) |
| Cdkn2a (p16) Fwd | 5′-CGTACCCCGATTCAGGTGAT-3′ (3) |
| Cdkn2a (p16) Rev | 5′-TTGAGCAGAAGAGCTGCTACGT-3′ (4) |
| Mcp1 Fwd | 5′-GCATCCACGTGTTGGCTCA-3′ (5) |
| Mcp1 Rev | 5′-CTCCAGCCTACTCATTGGGATCA-3′ (6) |
| Gapdh Fwd | 5′-AAGGTCATCCCAGAGCTGAA-3′ (7) |
| Gapdh Rev | 5′-CTGCTTCACCACCTTCTTGA-3′ (8) |

Multiplex Analysis of SASP Factors.

Serum levels of MCP-1, IL-6, and TNFα were analyzed in a multiplex assay using the Milliplex Map Mouse Metabolic Hormone Magnetic Bead Panel kit (Millipore Sigma, St. Louis, Mo.). 10 μL of serum was analyzed in duplicate (n=9-11 per group) and analyte concentrations were quantified on a Luminex 200 (Luminex Corporation, Austin, Tex.) microplate reader.

MCP-1 ELISA.

Serum concentrations of MCP-1 were surveyed for by mouse MCP-1 specific ELISA (Raybiotech, Norcross, Ga.). 50 μL of serum, obtained at the time of sacrifice, was used for ELISA per manufacturer's specification and absorbance was quantified at 450 nm using a Spectramax i3 (Molecular Devices, Sunnyvale, Calif.) plate reader. All standards and samples were measured in duplicate (n=5-6 animals per group except Bubr1^{H/H} and C57BL/6 controls where n=3 was used). 100 μL of conditioned media (48 h after splitting cells) from MEFs (n=3 per group) was used for the analysis of MCP-1 concentration by ELISA.

Pharmacologic Interventions.

Two year old C57BL/6J mice were obtained from the NIA Rodent Colony and treated with encapsulated rapamycin (Rapamycin Holdings, San Antonio Tex.) at 14 ppm for females and 42 ppm for males, or control (encapsulated only) diet for 8 weeks. Mice were sacrificed at 26 months and serum collected preceding necropsy. Ercc1^{-/Δ} mice were treated with Dasatinib and Quercetin as described elsewhere (see, e.g., Zhu et al., 2015 Aging cell. 14(4):644-658). Briefly, Ercc1^{-/Δ} mice were treated weekly with a combination of Dasatinib (5 mg/kg) and Quercetin (50 mg/kg) starting at 4-6 weeks of age. Therapeutics were administered in 10% PEG400 by oral gavage. Litters with multiple Ercc1^{-/Δ} mice were used to enable comparison of sibling pairs that were treated with therapeutic vs vehicle only. Mice (n=5-6 per group) were sacrificed at 16 weeks and serum collected by cardiac puncture.

Kidney Histology Assessment.

Mouse tissues were collected at necropsy and placed in 10% buffered formalin for 48 hours, transferred to 70% alcohol, and subsequently processed into paraffin blocks for sectioning and hematoxylin and eosin staining. Histology slides were validated for age-related lesions by a veterinary pathologist and scored for lesion severity to create a composite lesion score for age-related renal pathology in each animal (see, e.g., Ladiges 2016 Pathobiol Aging Age Relat Dis. 6:31478).

Participants and Research Protocol.

Human procedures and the use of biospecimens were approved by the Mayo Clinic Institutional Review Board. For comparison of MCP-1 levels with frailty, a sample of 63 individuals (27 women and 36 men) age 65 years or older diagnosed with severe aortic stenosis and scheduled for surgical or transcatheter aortic valve replacement were recruited between July 2013 and May 2015 to the Mayo Clinic in Rochester, Minn. (Schafer 2016 Cell Metab. 23(6):1207-15). Frailty assessment was conducted prior to surgery and was based upon the CHS criteria, defined by the following metrics: weak grip strength by electronic dynamometer (less than 17-21 kg for women and 29-32 kg for men, normalized to BMI), slow walk speed by a handheld ultrasonic monitor (less than 0.83 meters per second), self-report of low endurance and energy on the Center for Epidemiological Studies Depression Scale (self-report of exhaustion), unintentional weight loss (greater than or equal to 10 pounds in the prior year), and low physical activity by the Physical Activity Scale for the Elderly (men, less than 383 kcal expended per week; women, less than 270 kcal expended per week) (Fried et al., 2001 J Gerontol A Biol Sci Med Sci. 56(3):M146-56). Fasted blood samples were collected in EDTA at the time of surgery and were centrifuged and stored at −80° C. A Procartaplex Luminex immunoassay (Affymetrix eBioscience, San Diego, Calif.) was used for plasma MCP-1 quantification, according to manufacturer's specifications. For comparison of MCP-1 levels with age, plasma samples from 280 participants age 20-90, evenly distributed by decade and sex, were analyzed. Sampled participants were representative of the general population of Olmsted county. Selection criteria was defined as BMI: males 18.5-35 and females 18.5-40 with no history of cancer before age 50 (non-inclusive of breast or melanoma) or autoimmune disease.

Cytokine and Chemokines in Human Serum from the HANDLS Study.

Serum inflammatory markers and cytokines (FIG. 4) were quantified using Searchlight protein arrays from Aushon Biosystems (Billerica, Mass.) from a sub-cohort of participants from the Healthy Aging in Neighborhoods of Diversity across the Life Span (HANDLS) study of the National Institute on Aging Intramural Research Program. Participants provided written informed consent and the study is approved by the Institutional Review Board of the National Institute on Environmental Health Sciences, NIH. The coefficient of variance was calculated for the measured markers from a sub-cohort of 78 women. Details about this sub-cohort have been described previously (Noren Hooten et al., 2012 Arterioscler Thromb Vasc Biol. 32:2776-2784).

Statistical Methods.

For mouse data analyses, unpaired t and one-way ANOVA tests were used to compare MCP-1 concentrations between sex, genotype, and drug intervention groups. Linear regression analysis was used to summarize the relationship between MCP-1 and age in wild-type mice. For human data analyses, unpaired t, Mann-Whitney, and Chi-square tests were used to compare demographic and anthropometric variables. Linear regression models were used to summarize relationships between MCP-1 and age, sex, and combined age and sex. Natural log transformations or non-parametric tests were applied for non-normally distributed variables as indicated. Analyses were performed using R 3.3.2, JMP Pro 10, and GraphPad Prism 7.

Example 2 Fisetin is a senolytic drug—a drug that kills senescent cells. Genetic or pharmacologic ablation of senescent cells has been demonstrated to extend the health of progeroid or aged wild-type mice.

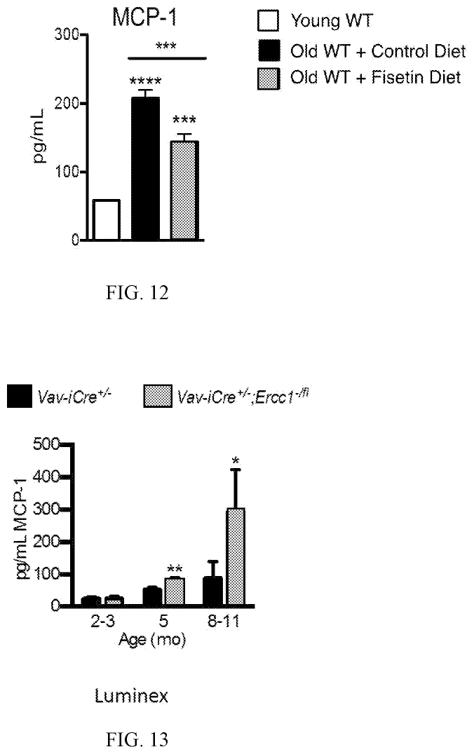

At 85-weeks of age (>20 mth), male and female mice were administered a diet containing 500 ppm (500 mg/kg) fisetin or fed a control diet with no drug. n=8-9 mice per group. In the treatment group, median lifespan was increased 17% and maximum lifespan increased 12%. Log rank (Mantel-Cox) test. Tissue function was improved and age-related histopathology was decreased in multiple organs. Levels of MCP-1 were measured in the serum of these mice by ELISA. n=5 mice per group. One-way ANOVA with Tukey's multiple comparison test. Values represented as the mean SEM, ***p<0.001, ****p<0.0001. Therefore, circulating levels of MCP-1 correlate with drug-induced improvements in health and lifespan (FIG. 12).

Vav-iCre^{+/-};Ercc1^{-/fl} mice age prematurely and have an increased abundance of senescent cells in multiple tissues compared to age-matched control mice due to deletion of the DNA repair gene Ercc1 in hematopoietic cells. Accordingly, MCP-1 levels are significantly greater in the serum of Vav-iCre^{+/-};Ercc1^{-/fl} mice compared to age-matched control mice (FIG. 13). MCP-1 levels were measured in the serum of Vav-iCre^{+/-};Ercc1^{-/fl} mice and sibling controls using the Luminex platform. n=6-10 mice per group. Values represent mean±SD. Two-tailed unpaired Student's t test. *p<0.05, **p<0.01.

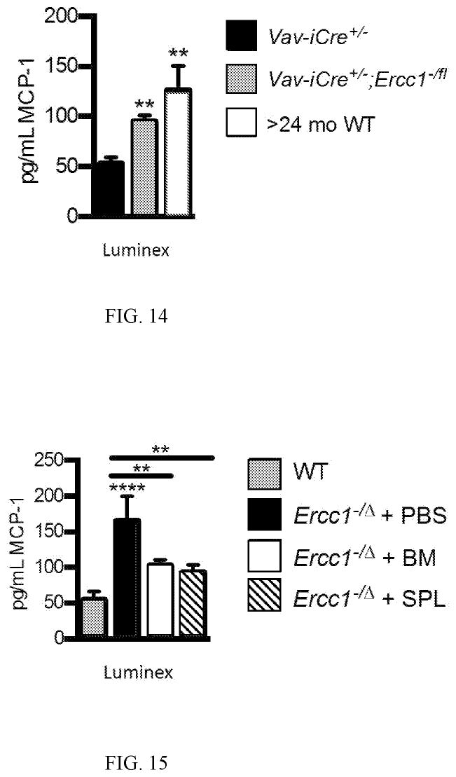

Transplantation of immune cells from aged mice can drive aging in recipient mice. To assess this, splenocytes isolated from 8-10-month-old Vav-iCre^{+/-} (control), 8-10-month-old Vav-iCre^{+/-};Ercc1^{-/fl} (prematurely aged), and 2+-year-old (aged normally) mice were retro-orbitally injected into 3-4-month-old p16^{Ink4+/Luc} mice (n=3-4 recipient mice per group). Luminescence (p16 expression) was increased significantly in recipient mice transplanted with old WT or Vav-iCre^{+/-};Ercc1^{-/fl} immune cells, indicating that the immune cells drive senescence and aging in trans (FIG. 14). Circulating MCP-1 levels in recipient were measured 15 days post-injection. MCP-1 levels were significantly increased in recipient mice transplanted with “aged” immune cells. Values represent mean±SD. One-way ANOVA with Tukey's test. **p<0.01.

Transplantation of immune cells from young healthy mice can slow aging in recipient mice. To prove this, 3-month-old progeroid Ercc1^{-/Δ} mice (n=4) were injected with either bone marrow cells or splenocytes from 2-month-old WT mice or phosphate buffered saline vehicle. One month later, the mice were euthanized and tissues were collected for analysis. Adoptive transfer of bone marrow cells and splenocytes suppressed expression of senescence markers, progeroid symptoms and age-related histopathology. Circulating level of MCP-1 correlated with this. In the progeroid Ercc1^{-/Δ} mice, levels of serum MCP-1 were significantly elevated compared to age-matched WT mice. Adoptive transfer suppressed serum MCP-1 back to baseline levels (FIG. 15). Values represent mean±SD. One-way ANOVA with Tukey's test. **p<0.01, ***p<0.001, ****p<0.0001.

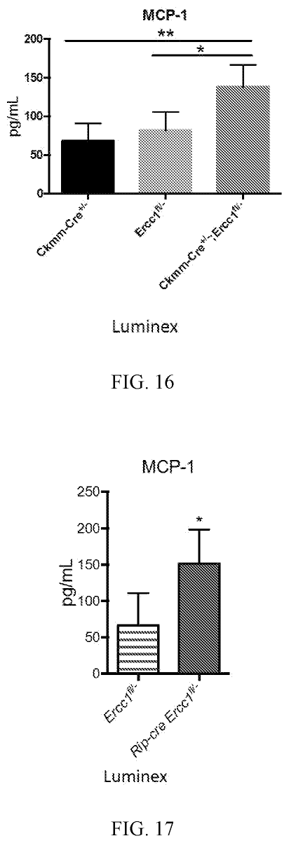

Ckmm-Cre^{+/-};Ercc1^{-/fl} mice are a murine model of congestive heart failure. These mice develop dilated cardiomyopathy by 5 months of age. They die a sudden death due to cardiac arrest before 7 months of age. MCP-1 levels in the serum of 4-6 mo Ckmm-Cre^{+/-}; Ercc1^{-/fl} mice is significantly higher than that of age-matched controls (FIG. 16). Thus, MCP-1 levels correlate with the presence of an age-related disease. n=4-5 mice per group. Values represent mean±SD. One-way ANOVA with Tukey's test. *p<0.05, **p<0.01.

Rip-cre Ercc1^{-/fl} mice are a model of Type II diabetes mellitus. The animals lose glucose homeostasis by 3 months of age due to loss of beta cells and beta cell function. Male mice become morbidly obese by 5 months of age. MCP-1 levels in the serum of 6-9 month-old Rip-cre Ercc1^{-/fl} mice is significantly elevated compared to age-matched controls (FIG. 17). Thus, MCP-1 levels correlate with the presence of a second age-related disease. n=5-12 mice per group. Values represent mean±SD. Two-tailed unpaired Student's t test. *p<0.05.

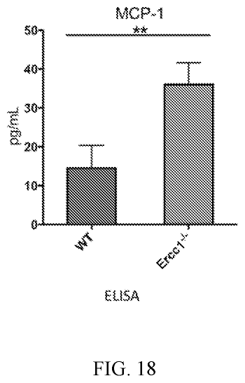

Reduced expression of ERCC1-XPF in humans and mice causes premature aging. Specifically in the brain, there is premature cerebral atrophy and cognitive decline. Primary neurons were isolated from day 15 WT and Ercc1^{-/-} embryos (n=3-4 per group) and cultured for 5 days. Conditioned media was collected and MCP-1 concentrations measured. The neurons isolated from diseased brains secreted significantly higher levels of MCP-1 (FIG. 18). Values represent mean±SD. Two-tailed unpaired Student's t test. **p<0.01.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-16. (canceled)

17. A method for slowing the progression of biological aging, wherein said method comprises administering a composition comprising a senolytic agent to a mammal identified as having an elevated level of circulating MCP-1 as compared to an average circulating MCP-1 level of healthy control mammals of the same chronological age as said mammal.

18. The method of claim 17, wherein said mammal is a human.

19. The method of claim 18, wherein said human has a chronological age that is over 55 years.

20. The method of claim 17, wherein said senolytic agent is fisetin.

21. The method of claim 17 any one of claims 17-20, wherein said composition comprises fisetin.

22. The method of claim 17 any one of claims 17-21, wherein said elevated level of circulating MCP-1 is at least about 10 percent greater than said average circulating MCP-1 level.

23. A method for slowing the progression of biological aging, wherein said method comprises administering a composition comprising a senolytic agent to a mammal identified as having a biological age based at least in part on an elevated level of circulating MCP-1, wherein said biological age is greater than the chronological age of said mammal.

24. The method of claim 23, wherein said mammal is a human.

25. The method of claim 24, wherein said human has a chronological age that is over 55 years.

26. The method of claim 23 any one of claims 23-25, wherein said senolytic agent is fisetin.

27. The method of claim 23 any one of claims 23-26, wherein said composition comprises fisetin.

28. The method of claim 23 any one of claims 23-27, wherein said biological age is at least about 10 percent greater than said chronological age.


SEQUENCE LISTING (8 sequences)

SEQ ID NO: 1 - DNA, 20 bp, Mus musculus
gccttagccctcactctgtg

SEQ ID NO: 2 - DNA, 20 bp, Mus musculus
agctggccttagaggtgaca

SEQ ID NO: 3 - DNA, 20 bp, Mus musculus
cgtaccccgattcaggtgat

SEQ ID NO: 4 - DNA, 22 bp, Mus musculus
ttgagcagaagagctgctacgt

SEQ ID NO: 5 - DNA, 19 bp, Mus musculus
gcatccacgtgttggctca

SEQ ID NO: 6 - DNA, 23 bp, Mus musculus
ctccagcctactcattgggatca

SEQ ID NO: 7 - DNA, 20 bp, Mus musculus
aaggtcatcccagagctgaa

SEQ ID NO: 8 - DNA, 20 bp, Mus musculus
ctgcttcaccaccttcttga